(19) United States
(12) Patent Application Publication
(10) Pub. No.: US 2009/0214624 A1
(43) Pub. Date: Aug. 27, 2009

(54) TOPICAL NITRIC OXIDE DONOR DEVICES AND METHODS FOR THEIR THERAPEUTIC USE

(75) Inventors: Daniel J. Smith, Stow, OH (US); Marco Lopez, Wauwatosa, WI (US); Patricio Lopez-Jaramillo, Santander (CO)

Correspondence Address: ROETZEL AND ANDRESS, 222 SOUTH MAIN STREET, AKRON, OH, 44308, US

(73) Assignee: THE UNIVERSITY OF AKRON, Akron, OH (US)

(21) Appl. No.: 11/719,894

(22) PCT Filed: Nov. 29, 2005
(86) PCT No.: PCT/US05/43051

§ 371 Date: Apr. 13, 2009

Related U.S. Application Data
(60) Provisional application No. 60/631,454, filed on Nov. 29, 2004.

(51) Int. Cl. A61K 9/70; A61K 33/00; A61P 9/00

(52) U.S. Cl. 424/449; 424/718

(57) ABSTRACT

The present invention is related to topical nitric oxide delivery systems, and to using the same for mitigating or remediating various disease states The present invention is also related to using topical nitric oxide delivery systems for enhancing blood flow.

### FIELD OF THE INVENTION

The present invention relates to devices and methods for topical delivery of nitric oxide for therapeutic purposes.

### BACKGROUND OF THE INVENTION

The need for the present invention arises from the clinical necessity to direct doses of therapeutic agents such as nitric oxide to particular tissues. Specifically in regard to nitric oxide, it is especially important to direct doses to the areas of the patient where dosing is indicated because of the high reactivity of nitric oxide radicals. Conditions that can benefit from topical nitric oxide therapy include, without limitation, diabetic ulcers, peripheral neuropathy resulting from diminished blood flow, cold hands and feet syndromes, and transdermal drug delivery where the diffusion rate is slow.

Until now, the art has been deficient in devices and methods for enhancing blood flow with topical nitric oxide therapy. The present invention addresses this gap in the art by providing devices and methods for topically applying nitric oxide releasing compounds for enhancing blood flow.

### SUMMARY OF THE INVENTION

The present invention is directed to nitric oxide-releasing dressings and compositions and their use for mitigating or remediating diseases including peripheral neuropathy. Furthermore, the present invention is directed to the use of a transdermal patch containing agents for nitric oxide delivery.

The present invention is also directed to a therapeutic method comprising the steps of placing a nitric oxide delivering patch in contact with an area of the body where blood flow is to be enhanced, activating said patch, and delivering nitric oxide transdermally.

Additionally, the present invention is directed to a topical nitric oxide deliver device comprising a nitric oxide delivering patch capable of including nitric oxide derivative moieties, wherein the patch is capable of being placed in contact with an area of the body where blood flow is to be enhanced, at least one nitric oxide derivative moiety, and an activating agent, wherein the activating agent is either a component of the device or added to the device during treatment.

The present invention is also directed to a means for enhancing blood flow, and a means for remediating disease resulting from vascular insufficiencies.

### BRIEF DESCRIPTION OF THE DRAWINGS

FIG. 1 is a schematic representation of the present invention as it relates to a patch containing ascorbic acid activator and a nitric oxide donor, which are spatially separated until contacted by water,

FIG. 2 is a water-activated nitric oxide donor separated from the skin by a water-impermeable membrane,

FIG. 3 is a pair of before and after photographs showing the results of treating a diabetic ulcer with the present invention, and

FIG. 4 is a pair of before and after photographs showing the results of treating a diabetic ulcer, located on a foot, with the present invention.

### DETAILED DESCRIPTION OF ILLUSTRATIVE EMBODIMENTS

The present invention is directed to devices and methods for using nitric oxide-releasing compounds to mitigate or remediate diseases including peripheral neuropathy. More particularly, the present invention is directed to transdermal patches containing agents for nitric oxide delivery, and a variety of topical delivery systems such as creams. Furthermore, the present invention is directed to methods of using the topical nitric oxide delivery systems taught herein.

Transdermal patch devices within the scope of the present invention generally comprise at least one fibrous component, which contains a nitric oxide-releasing agent. These agents are substantially inactive until contacted with a Bronsted acid known as an activating agent, which includes without limitation protic liquids such as water, physiological buffers, body fluids, saline and the like. Upon activation, the nitric oxide donor components of the patch begin to produce nitric oxide, which diffuses into and through the skin. Upon penetrating the skin, nitric oxide causes capillary beds in the vicinity of the patch to engorge with blood thereby increasing blood flow to the tissues in that area. Surprisingly, it has been discovered that delivering nitric oxide in this manner mitigates or remediates various pathological conditions including peripheral neuropathy.

Generally, the fibrous component of a transdermal patch embodiment may comprise any fiber capable of containing one or more nitric oxide-releasing agents. Fibers within the scope of the present invention include, without limitation, those which reversibly react with nitric oxide to form functional groups, located on the fiber, that amount to nitric oxide-releasing agents; and, those which otherwise sequester or immobilize nitric oxide-releasing agents. More particularly, fibers that are suitable for derivatizing tend to contain secondary amine moieties inasmuch as secondary amines are known to react with nitric oxide to form diazeniumdiolates, which undergo a first order reaction evolving nitric oxide (I-II).

### (I) PEI Structure

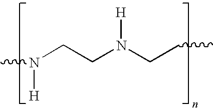

### (II) Derivatized PEI Diazeniumdiolate Structure

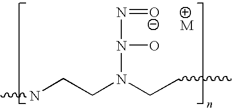

Acceptable fibrous components include any fiberizable material comprising secondary amine moieties. More particularly, acceptable fibrous components comprise polymers including but not limited to polyethyleneimine, polypropyleneimines, polybutyleneimines, polyurethanes, polyamides, and both linear and branched forms of each of the foregoing polymers. Additionally, fibrous components within the scope of the present invention include, without limitation, any copolymer of the foregoing polymers, and any combination thereof. More particularly, acceptable fibrous components include any of the foregoing polymers grafted to an inert backbone, e.g. polyethyleneimine grafted to an otherwise relatively inert backbone such as a polysaccharide backbone, especially a cellulosic backbone. An example of a material for forming a fibrous component in accordance with the present invention is high density linear polyethyleneimine (I-II) having a weight average molecular weight of greater than about 200,000. Linear PEI is soluble in common solvents such as ethanol, but is insoluble in water.

The fibrous component may be formed by electrospinning and by the Nanofibers by Gas Jet (NGJ) processes, which are known in the art. Briefly, the NGJ method comprises using a device having an inner tube and a coaxial outer tube with a sidearm. The inner tube is recessed from the edge of the outer tube thus creating a thin film-forming region. Polymer melt is fed in through the sidearm and fills the empty space between the inner tube and the outer tube. The polymer melt continues to flow toward the effluent end of the inner tube until it contacts the effluent gas jet. The gas jet impinging on the melt surface creates a thin film of polymer melt, which travels to the effluent end of tube where it is ejected forming a turbulent cloud of nanofibers.

It will also be appreciated by those skilled in the art that the fibrous component may be formed in accordance with the present invention by methods other than electrospinning or NGJ. Any method of forming organic polymers into fibers known in the art may be used. For instance, extrusion methods such as wet spinning, dry spinning, melt spinning, and gel spinning are all acceptable methods of forming fibers in accord with the present invention. Generally, finer denier fibers yield fiber mats having greater surface area and thus more nitric oxide-releasing agents, which generally results in better performance. Accordingly, electrospinning and NGJ are especially effective methods for manufacturing the fibrous component.

In general the present invention may take the form of a nonwoven fiber mat. The present invention may also take the form of a layered nonwoven fiber mat. The present invention may further comprise one or more fibrous layers bounded by a membrane that selectively passes nitric oxide while being impervious to liquids such as activator solutions. However, the present invention need not include fibers at all. As such, alternative embodiments include a cream, gel, salve, ointment, balm, cerate, demulcent, liniment, lotion, lenitive, and/or unguent containing NO-releasing components. For instance, such embodiments can comprise a NO-releasing component and an activating component, which are mixed just prior to, or during, application.

In general terms, the nitric oxide-releasing agent of the present invention comprises any chemical entity that yields nitric oxide when stimulated to do so by an activator of the present invention. It is appreciated in the art that these agents can take several forms including, but not limited to, diazeniumdiolates. It is further appreciated in the art that nitric oxide-releasing agents can take the form of O-alkylated diazeniumdiolate, or any O-derivatized diazeniumdiolate where the O-derivative can be converted back to the diazeniumdiolates. Such O-derivatized diazeniumdiolates are generally more stable than salts. Particularly, the energy of activation of the decomposition reaction is generally substantially higher than that of the non-O-derivatized form. Thus, the derivative tends to either not evolve nitric oxide in the absence of an enzymatic activator, or to extend the half-life of the diazeniumdiolate significantly. The non-O-derivatized diazeniumdiolate functional group, such as a salt, is an effective nitric oxide-releasing agent for the present invention, and is known to decompose by a first order mechanism in the presence of a proton source, i.e. activators.

Reacting PEI with nitric oxide ordinarily results in the formation of diazeniumdiolates, which causes the PEI to lose its solubility in ethanol, and in most cases become insoluble in water. When nitric oxide-modified PEI polymers are exposed to water they begin to decompose in predictable ways resulting in the release of nitric oxide. A typical nitric oxide release profile from a PEI fiber mat is generally short, one to two days being a representative time.

Other nitric oxide-releasing agents within the scope of the present invention include, without limitation, nitrites such as nitro-functionalized compounds. Representative compounds include nitroglycerine, N-(Ethoxycarbonyl)-3-(4-morpholinyl)sydnoneimine; 3-morpholinosydnonimine; 1,2,3,4-Oxatriazolium; 5-amino-3-(3,4-di-chlorophenyl)-chloride; 1,2,3,4-Oxatriazolium; 5-amino-3-(chloro-2-methyl-phenyl)chloride; 1,2,3,4-Oxatriazolium, 3-(3-chloro-2-methylphenyl)-5-[[[cyanomethylamino]carbonyl]amino]-hydroxide inner salt; S-nitroso-N-acetyl-(D,L)-penicillamine; 1-[(4′,5′-Bis(carboxymethoxy)-2′-nitrophenyl)methoxy]-2-oxo-3,3,diethyl-1-triazene dipotassium salt; and [1-(4′,5′-Bis(carboymethoxy)-2′-nitropheyl)methoxy]-2-oxo-3,3-diethyl-1-triazine diacetoxymethyl ester.

Generally, activators comprise any compound that stimulates the nitric oxide-releasing agent to produce nitric oxide. Where a diazeniumdiolate is the agent, acceptable activators comprise proton sources, i.e. Bronsted acids. Representative activators comprise water, body fluids such as blood, lymph, bile and the like; and methanol, ethanol, propanols, butanols, pentanols, hexanols, phenols, naphthols, polyols, and the like. Further activators within the scope of the present invention comprise common aqueous acidic buffers including, without limitation, phosphates, succinates, carbonates, acetates, formates, propionates, butyrates, fatty acids, and amino acids, ascorbic acids and the like. Activators also include, without limitation, water, body fluids such as blood or lymph, alcohols, and common aqueous acidic buffer solutions. Other activators within the scope of the present invention include enzymatic and other catalytic compounds.

Methods of activating the nitric oxide donors of the present invention include, without limitation, contacting with an activator. Activators, include compounds such as water, saline, alcohols, protic solvents, solutions of ionic materials such as salts including organic and/or inorganic salts, and solutions of organic acids such as ascorbic acid and inorganic acids, physiological buffers, and the like. Further activation methods consistent with the present invention include, without limitation, photonic activation, wherein exposure to electromagnetic radiation stimulates a chemical reaction, which produces nitric oxide.

In one embodiment the present invention comprises a nonwoven nanofibrous mat derivatized with nitric oxide so as to form diazeniumdiolate moieties. The invention, in this form, could be activated by applying the mat to an open wound, and allowing the body fluids exuded by the wound activate nitric oxide production. Another embodiment comprises the foregoing wherein the mat is a component of an adhesive bandage.

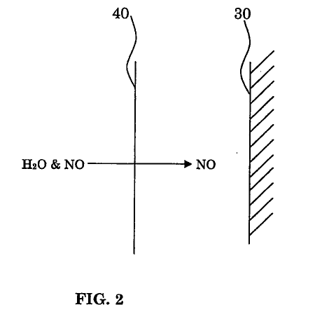

Another embodiment of the present invention may comprise multiple nanofibrous mat layers. For instance, one layer could comprise a nitric oxide-yielding fibrous material, while another layer comprises a fibrous material soaked in activator solution, wherein the two layers are separated by a barrier layer. The dressing is activated when the barrier layer is breached, for instance, by rupturing it. An additional embodiment comprises the foregoing, wherein the nitric oxide-yielding layer and the activator layer are substantially enclosed within an outer liquid barrier membrane 40, at least a portion of which is permeable to nitric oxide (FIG. 2).

Still another embodiment comprises a nitric oxide derivatized nanofiber electrospun directly onto a wound or other area where nitric oxide dosing would be beneficial. In this arrangement the fluids exuded by the wound can serve as the activator. Alternatively, an activator could be added to the mat after electrospinning.

Uses of the present invention include, without limitation, (1) raising blood flow to damaged tissues to promote healing; (2) raising blood flow to areas of the body having a cold sensation due to poor circulation; (3) delivering NO to areas of the body experiencing neuropathy; and (4) raising blood flow to a locus of transdermal drug delivery for the purpose of accelerating the delivery. The latter use may be particularly helpful when transdermal drug delivery is otherwise slow. Accordingly, the present invention could be employed to enhance drug delivery by its use in conjunction with drugs the delivery of which would benefit from increased blood flow. In any case, the present invention may be applied regardless of whether blood flow is otherwise elevated, normal or sub-normal. Still further uses of the present invention include treatment of persistent sores related to diabetes, or leishmania.

A dosage rate of nitric oxide in accordance with the present invention is from about 5 nmol/cm² to about 100 μmol/cm² over the course of six hours. Other dosage rates of nitric oxide in accordance with the present invention are about 5 μmol/cm² over the course of six hours, and about 10 μmol/cm² over the course of 24 hours.

In order to demonstrate the practice of the present invention, the following examples have been prepared. The examples should not, however, be viewed as limiting the scope of the invention. The claims will serve to define the invention.

### EXAMPLES

### Example 1

In one example of the present invention a male diabetic patient having a persistent open sore on his foot is treated with the present invention. In addition to the tissue damage to the skin and underlying muscle, the patient suffers from neuropathy induced by poor circulation resulting from diabetes. As a result of the treatment the sore closes and the patient regains sensation in his foot as well. That is to say, he experiences a reversal of his circulation-induced neuropathy.

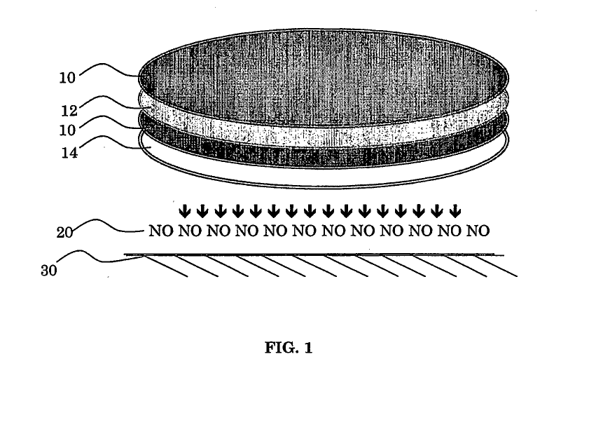

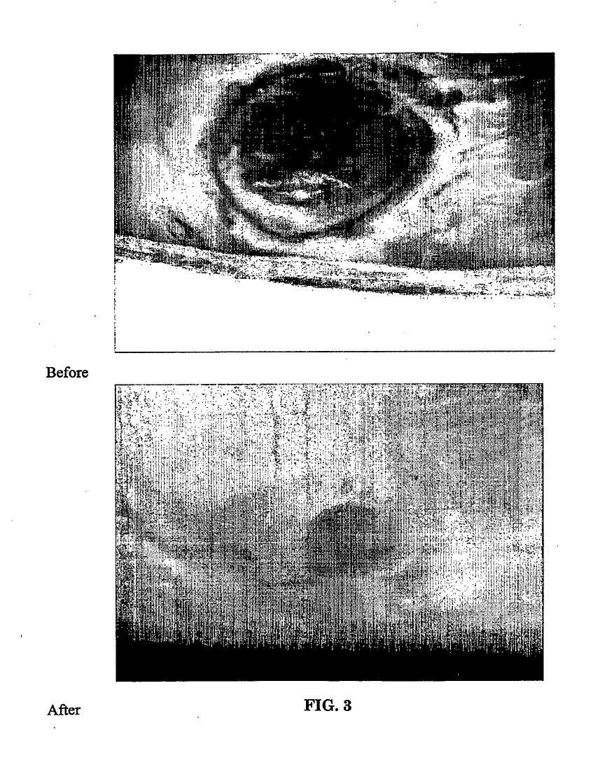

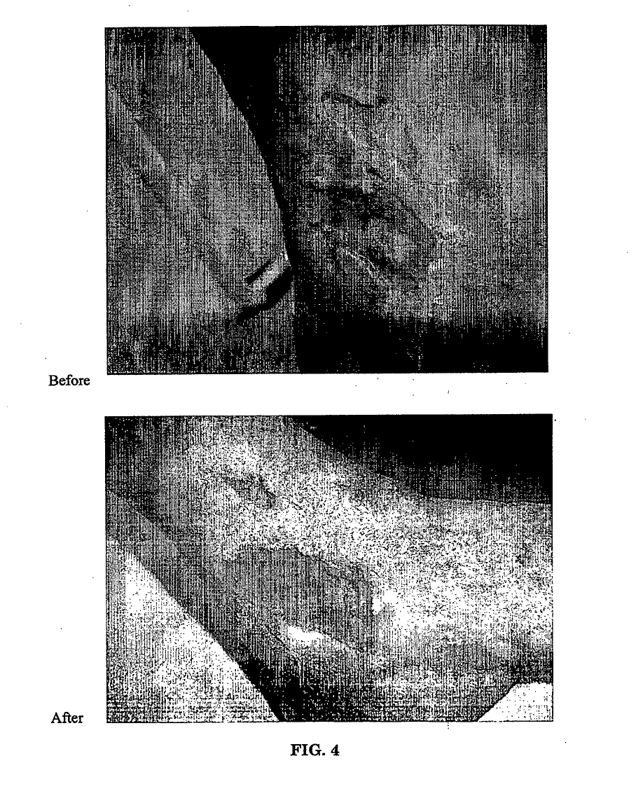

In this instance the present invention takes the form of a patch consistent with the drawing in FIG. 1, and therapeutic use thereof. The patch comprises four layers. The top layer, which is farthest from the skin, is a super-absorbent fibrous polymer 10. The next layer 12 comprises a fibrous polymer, which is a polyurethane commercially available under the trade name TECOPHILIC, and also contains ascorbic acid; however, any other weak acid activator would also be acceptable. The third layer is essentially the same as the first 10. The fourth layer 14, which is in contact with the skin, comprises polymers commercially available under the trade names TECOPHILIC and DOWEX, which are derivatized with a diazeniumdiolate compound, or a nitrite inorganic salt. The present invention is inactive until activated by contacting it with water or aqueous fluids, whereupon the nitrite compound yields nitric oxide 20. Nitric oxide 20, produced in this way, diffuses through the skin 30 and stimulates blood flow in the vicinity of the patch. After a period of weeks, the enhanced blood flow results in healing, as shown in FIGS. 3 and 4.

Various modifications and alterations that do not depart from the scope and spirit of this invention will become apparent to those skilled in the art. This invention is not to be limited to the illustrative embodiments set forth herein.

We claim:

1. A therapeutic method comprising the steps of:
placing a nitric oxide delivering transdermal patch in contact with an area of the body where blood flow is to be enhanced;
activating the transdermal patch; and
delivering nitric oxide transdermally.

2. The method of claim 1, wherein the step of placing further comprises placing a fibrous material.

3. The method of claim 2, wherein the step of placing further comprises placing a nanofibrous material.

4. The method of claim 2, wherein the step of placing further comprises placing a nitric oxide derivatized material.

5. The method of claim 2, wherein the step of placing further comprises placing a diazeniumdiolate material.

6. The method of claim 2, wherein the step of placing further comprises placing a nitric oxide derivatized polymer selected from linear polyethyleneimine, branched polyethyleneimine, any copolymer of polyethyleneimine, and any combination thereof.

7. The method of claim 2, wherein the step of placing further comprises a step selected from placing an adhesive bandage, placing a nonwoven mat, placing a woven mat, placing a gauze, and placing a layered mat.

8. The method of claim 1, wherein the step of placing further comprises spinning fibers directly onto the patient.

9. The method of claim 8 wherein the step of spinning further comprises a method selected from electrospinning, nanofibers by gas jet, wet spinning, dry spinning, melt spinning and gel spinning.

10. The method of claim 1, wherein the method is for treating neuropathy or a disease state resulting from vascular insufficiencies.

11. The method of claim 1, wherein the method is for treating a disease state resulting from vascular insufficiencies due to diabetes, or low blood pressure.

12. The method of claim 1, wherein the method is for accelerating delivery of a therapeutic agent.

13. The method of claim 12, wherein the method is for accelerating transdermal delivery of a drug.

14. The method of claim 1, wherein practicing the method results in mitigated neuropathy.

15. The method of claim 1, wherein practicing the method results in remediated neuropathy.

16. The method of claim 1, wherein the activating agent is selected from ascorbic acid, water, body fluids, lymph, bile, methanol, ethanol, propanol, butanol, pentanol, hexanol, phenol, naphthol, phosphate, succinate, carbonate, acetate, formate, propionate, butyrate, fatty acids, and amino acids.

17. A means for enhancing blood flow.

18. A means for remediating neuropathy or a disease state resulting from vascular insufficiencies.

19. A topical nitric oxide delivery device comprising:
a nitric oxide delivering patch capable of including nitric oxide derivative moieties, wherein the patch is capable of being placed in contact with an area of the body where blood flow is to be enhanced;
at least one nitric oxide derivative moiety; and
an activating agent, wherein the activating agent is either a component of the device or added to the device during treatment.

20. The device of claim 19, wherein the patch further comprises a fibrous material.

21. The device of claim 19, wherein the patch further comprises a nanofibrous material.

22. The device of claim 19, wherein the patch further comprises a nitric oxide derivatized material.

23. The device of claim 19, wherein the patch further comprises a diazeniumdiolate material.

24. The device of claim 19, wherein the patch further comprises a nitric oxide derivatized polymer selected from linear polyethyleneimine, branched polyethyleneimine, any copolymer of polyethyleneimine, and any combination thereof.

25. The device of claim 19, wherein the patch further comprises a component selected from an adhesive bandage, a nonwoven mat, a woven mat, a gauze, and a layered mat.

26. The device of claim 19, wherein the patch further comprises fibers spun directly onto the patient.

27. The device of claim 19, wherein the activating agent is selected from ascorbic acid, water, body fluids, lymph, bile, methanol, ethanol, propanol, butanol, pentanol, hexanol, phenol, naphthol, phosphate, succinate, carbonate, acetate, formate, propionate, butyrate, fatty acids, and amino acids.

28. A topical nitric oxide delivery composition comprising:
a nitric oxide releasing component; and
an activating component, wherein the releasing component and activating component comprise a composition selected from a cream, a gel, a salve, an ointment, a balm, a cerate, a demulcent, a liniment, a lotion, a lenitive, and an unguent.
